(19) United States
(12) Patent Application Publication
(10) Pub. No.: US 2024/0090766 A1
(43) Pub. Date: Mar. 21, 2024

(54) Ophthalmic Device with Self Alignment for Operator-Less Operation

(71) Applicant: Twenty Twenty Therapeutics LLC, South San Francisco, CA (US)

(72) Inventors: Supriyo Sinha, Menlo Park, CA (US); Dimitri Azar, Chicago, IL (US)

(21) Appl. No.: 18/465,913

(22) Filed: Sep. 12, 2023

Related U.S. Application Data
(60) Provisional application No. 63/376,187, filed on Sep. 19, 2022.

(51) Int. Cl. A61B 3/15; A61B 3/00; A61B 3/113; A61B 3/16; A61B 3/18

(52) U.S. Cl. CPC A61B 3/152; A61B 3/0033; A61B 3/113; A61B 3/165; A61B 3/18

(57) ABSTRACT

A processor signals a display to show a target. Optics is positioned in an optical path taken by the target, as shown by the display, from the display to the eye of a user. The optics is configured to change accommodation by the eye and has adjustable focal length that enables the user to see the target with changing acuity. The processor then obtains an indication that the eye of the user is focused on the target. In response, the processor signals an alignment mechanism to align a sensor in the device to the eye of the user. After signaling the alignment mechanism to align the sensor to the eye of the user, the processor obtains sensor data, produced by the sensor, which measures a property of the eye. Other aspects are also described and claimed.

### CROSS-REFERENCE TO RELATED APPLICATION

This nonprovisional patent application claims the benefit of the earlier filing date of U.S. Provisional Application No. 63/376,187, filed Sep. 19, 2022.

### FIELD

The subject matter of this disclosure relates to ophthalmic instruments for measuring properties of an eye of a user.

### BACKGROUND

There are several ophthalmic devices that measure properties of an eye of a user, such as intraocular pressure, a condition of the retina, topography of the cornea, etc. These devices may require alignment between the device and the user's eye. Traditionally, the alignment has been achieved based on input by a human (operator) such as a physician, a physician assistant or nurse who views the user's eye while the user's head is resting on a chin/head rest, where the operator manually moves the device into its proper position.

### SUMMARY

It would be desirable to have an ophthalmic device that can be positioned against an eye of a user while the device makes measurements of their eye, without requiring any assistance from an operator. This would reduce costs and enable the measurements to be taken outside of a physician's office. An aspect of the disclosure here is an ophthalmic device that presents a visual, alignment target at which the user is instructed to look. The user may be instructed to bring their eye into proximity of the device and then look for the target through a view port of the device. The target may be either a static image or a dynamic image that is presented by a display, e.g., a microdisplay, within the device. An alignment mechanism within the device automatically aligns an eye property measurement sensor in relation to the eye, to ensure accuracy of the measurements. The alignment is automatic in that it does not require input from an operator.

The alignment may require that the user can see the target with sufficient acuity. But many eye measurements do not allow the user to wear spectacles or contact lenses during the measurement, and the wide distribution of myopia and hyperopia in the population makes it difficult for all users to see the target at a high enough resolution (which is needed to ensure comfortable and timely alignment.) In addition, there are changes to accommodation range (the distance between the eye and the target for comfortable viewing) as a user ages, and so this also decreases the population that could see the target sufficiently well.

In accordance with one aspect of the disclosure here, a device for measuring eye properties has a device housing (e.g., that of a tabletop instrument or a handheld one) in which there is a sensor subsystem (sensor) that measures a property of the user's eye. An electronically controlled alignment mechanism to which the sensor is coupled serves to align the sensor to the eye of the user. Also in the device housing is a display that shows the target to the user's eye, and optics positioned in an optical path taken by the target (as it is being shown by the display) from the display to the eye of the user. The optics is configured to change accommodation by the eye, and it has adjustable focal length that enables the user to see the target with changing acuity. The alignment process performed by the alignment mechanism is triggered (to start or resume) in response to the processor obtaining an indication that the eye of the user is focused on the target (or that the user can see the target with sufficient acuity.) In this manner, the alignment which prepares the sensor to make a measurement of the eye property is likely to be faster and more accurate, which makes the eye examination process more efficient.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have advantages not specifically recited in the above summary.

### BRIEF DESCRIPTION OF THE DRAWINGS

Several aspects of the disclosure here are illustrated by way of example and not by way of limitation in the figures of the accompanying drawings, in which similar references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 shows a user holding in their hand an example, ophthalmic device against their eye, while the device is performing ophthalmic measurements on the user's eye.

FIG. 2 is a block diagram illustrating certain components of the example ophthalmic device of FIG. 1 that enable intraocular pressure, IOP, measurements.

FIG. 3 is a block diagram illustrating certain components of the example ophthalmic device of FIG. 1 that enable retina imaging or cornea topographic maps.

FIG. 4 is a flow diagram of a method performed by a programmed processor for measuring properties of the eye using an ophthalmic device.

### DETAILED DESCRIPTION

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

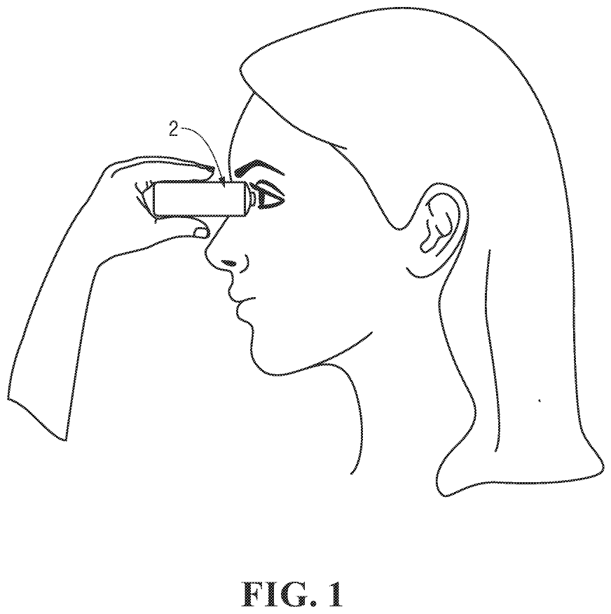

FIG. 1 shows a user holding in their hand an example, device 2 against their eye, while the device is performing ophthalmic measurements on the user's eye. Although the device 2 is illustrated as being a handheld device (where the user is holding the device housing in their hand), one alternative is a tabletop instrument where a device housing (of the device 2) has integrated therein or is attached to a stand, and where the stand can for example rest on a tabletop while the user brings their eye in proximity to the device 2. The proximity is a distance between the device housing and the eye at which a sensor 3 can be used to measure a property of the eye. The property of the eye being measured may be intraocular pressure, IOP, or it may be some aspect of the cornea or of the retina (e.g., as depicted in the form of a digital image or a map.)

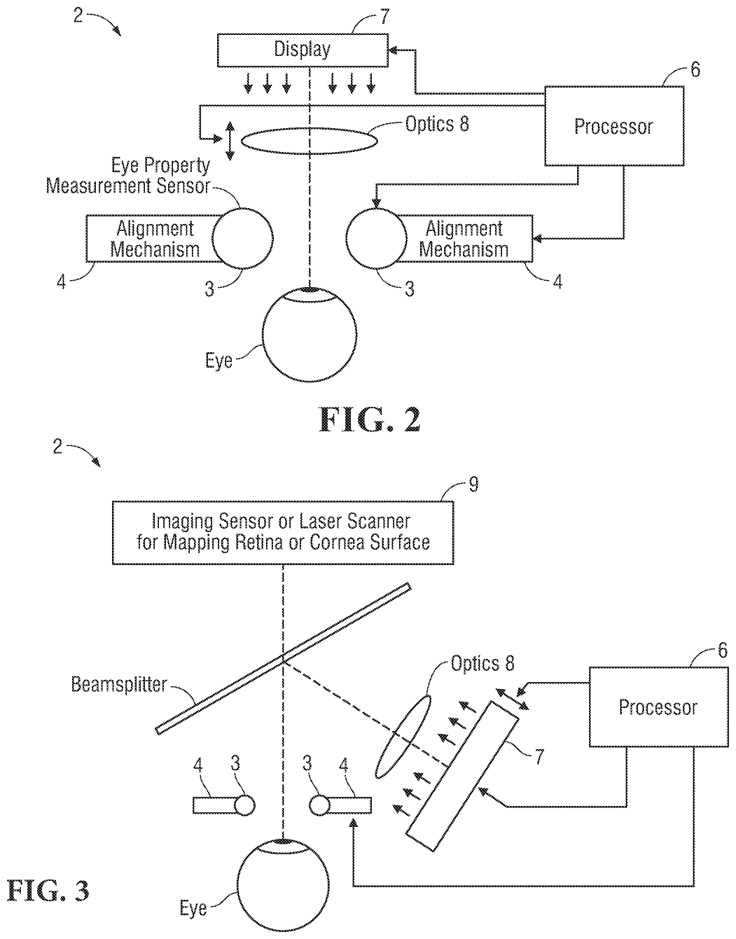

Referring now to FIG. 2, this is a block diagram illustrating several components of an example of the device 2, including the sensor 3 in the device housing. In one aspect, the sensor 3 may include a receiving element (e.g., an optical detector, a pixel array) as well as a transmitting element (e.g., an optical emitter), and any associated optical components (e.g., filters, lenses) that can be used to measure IOP. In that case, the sensor 3 may be an optical sensor that is part of air puff tonometer, where the sensor 3 is aimed at a particular region on the cornea surface while an air puff generator mechanism within the device 2 forces that region to flatten. The sensor 3 makes measurements of the cornea's flattened region, which measurements are then digitally processed to yield IOP values. In another aspect, the sensor 3 is part of an intraocular pressure monitoring subsystem that has a pressure measurement device implanted in the eye. There, the sensor 3 may be an optical receiver or transceiver that needs to be aligned with the implanted measurement device, to produce an TOP value.

The sensor 3 may need to be aligned with the eye in order to produce the measurement of the eye property. For that reason, the sensor 3 is coupled to an alignment mechanism 4 (that is also in the device housing.) The alignment mechanism 4 may include an actuator that is electronically controlled by a processor 6, and that serves to move the sensor 3, or in other words actuate any moveable component of the sensor 3 (e.g., an emitter, a detector, or an optical component of the sensor 3), on command by the processor 6.

The processor 6 is configured or programmed (e.g., when it is executing instructions stored in memory—not shown) to signal the alignment mechanism 4 to start or resume either an open loop or a closed loop process, to align the sensor 3 to the eye of the user. In one aspect of the disclosure here, the processor 6 may only do so in response to obtaining some indication that the eye of the user is focused on the target that is being displayed in the display 7, or in other words the user can see the target with sufficient acuity. This helps ensure that the alignment process correctly and quickly positions the sensor 3 for measuring the eye property. The target may be a graphical object, or an image of a real object, which is being shown by a display 7 in the device housing. The display 7 may be a microdisplay, e.g., a miniature display with a diagonal display size of less than 2 inches. The target may be either a static image or it may be an active image shown by the display 7. Status information may also be presented by the display 7, e.g., a countdown clock, which eye is being measured, etc. Optics 8 in the device housing is positioned in an optical path that is taken by the target (as the target is being shown by the display 7.) The optical path is from the display 7 to the eye of the user as depicted in the figures.

The optics 8 is configured to change accommodation by the eye and has adjustable focal length that enables the user to see the target with changing acuity. Changing accommodation lets the user see the target more easily, particularly in cases where the display 7 is positioned no more than two hundred millimeters from the eye (when the device housing is in proximity to the eye.) In one aspect, the optics 8 includes a convex solid lens that is motorized to be moveable as controlled electronically by the processor 6. That is a general description, of course, in that it covers cases where the optics 8 includes a series of two or more lenses (e.g., convex and concave) in which one or more of the lenses whose position in an axial direction can be adjusted and controlled electronically by the processor 6. The axial position could be adjusted by making the lens moveable in the axial direction, or equivalently making the display 7 moveable in the axial direction. In another aspect, the optics 8 includes a fluidic lens whose shape is controlled electronically by the processor 6 (to change the focal length.)

In one aspect, the processor 6 is configured to obtain the indication that the eye of the user is focused on the target, by prompting the user to indicate when the user can see the target clearly, while the processor 6 is signaling the optics 8 to change focal length. This prompting may be performed by the processor 6 signaling an audio subsystem (not shown) to instruct the user, “Please press the button or respond verbally, when you can see the target clearly.” The audio subsystem may have a microphone in the device housing, and the processor 6 processes an audio signal output by the microphone to detect audible input from the user as the indication that the user can see the target clearly.

As an alternative, the focal length of the optics 8 may be manually adjustable, by the user for example turning a knob in the device housing. The processor 6 in that case may also be configured to obtain the indication that the eye of the user is focused on the target, by receiving manual (e.g., a button press) or audible input from the user so that the user can see the target clearly. For instance, the user may be instructed to manually adjust the optics 8 using their fingers until they can see the target clearly at which point the user will press a button or speak a phrase which the processor 6 interprets as indicating that the user can see the target clearly.

In another aspect, the device 2 contains an eye tracking subsystem in the device housing. The processor 6 in that case is configured to process eye tracking data, produced by the eye tracking subsystem, to determine whether the eye is looking at the target that is being shown in the display 7, and in response the processor signals the alignment mechanism 4 to perform the alignment process.

In another aspect, referring now to FIG. 3, in addition to the sensor 3, there is an imager/scanner 9 within the device housing that can be used for mapping the retina or cornea of the eye. The imager/scanner 9 may include an imaging sensor as part of a still or video camera, a laser scanner, or both, together with their associated optics. The imager/scanner 9 are positioned to receive light that has reflected off the eye, from the back face of a beamsplitter. In contrast, the front face of the beamsplitter serves to reflect the light that is produced by the display 7 towards the eye so the user can see the target. The beamsplitter enables the device 2 to perform different eye property measurements: it enables the imager/scanner 9 to capture digital images of the eye's retina or cornea surface (which images may then be digitally processed before being displayed to a user as a picture of the retina, or a cornea topographic map); and it lets the user focus on a target in the display 7 during automatic alignment of the sensor 3 (which is measuring other eye properties, e.g., IOP.)

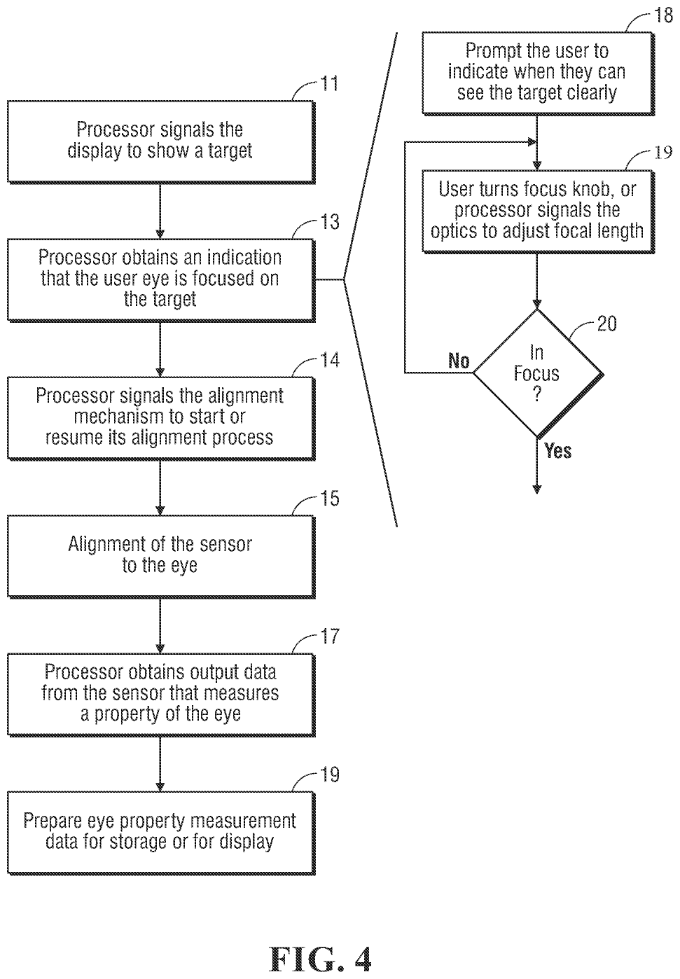

Referring now to FIG. 4, this is a flow diagram of a method performed by the processor 6 and by other components of the device 2, for measuring properties of the user's eye using the device 2. The method may begin with operation 11 in which the processor signals the display 7 to show a target. The user can then see the target through a view port of the device housing when their eye is positioned in proximity of the device 2 and is looking at the direction of the display 7. The coarsest level of alignment may be when the user can see the display 7 but is not focused on the target being shown in the display 7. A finer level of alignment is desired, which is when the user can see the target clearly (or is said to be “focused on” the target.) Thus, in operation 13, the processor 6 obtains an indication that the eye of the user is focused on the target. It then responds in operation 14 by signaling the alignment mechanism 4 to align the sensor 3 to the eye of the user. The alignment process then takes place as operation 15 (e.g., the alignment mechanism 4 adjusts a position of the sensor 3), and then once the sensor 3 and the eye are deemed to be aligned the processor 6, in operation 17, obtains sensor data, produced by the sensor 3, that measures some property of the eye (e.g., an IOP measurement.) In the case where the device 2 is equipped with the imager/scanner 9 as in FIG. 3, other eye property measurements can also be triggered now, such as taking a picture of the retina or producing a corneal topographic map. The processor 6 may then prepare the appropriate eye property measurement data from the sensor output data, for storage or for display to an operator (operation 19.)

In one aspect of the method in FIG. 4, operation 13, which involves determining when the eye of the user is focused on the target, includes operation 18. In operation 18, the processor 6 prompts the user to indicate when the user can see the target clearly. In the case where the optics 8 is electronically controllable, this may take place while or just before the processor signals the optics 8 to change focal length or the user is turning a manual focus knob (operation 19.) The prompt may be a spoken instruction, output by an audio subsystem of the device 2, that can be heard by the user. The user's response to the prompt is evaluated in operation 20, where the user's response may also be in audible form such as a phrase spoken by the user. The phrase may be recognized by the processor 6 processing an audio signal output by a microphone of the audio subsystem, e.g., “I can see the target clearly now.” If it is determined in operation 20 that the target is not in focus, then the method repeats with operation 19 by either waiting for the user to turn the focus knob or by the processor signaling the optics to adjust the focal length.

As an alternative to the processor 6 signaling a motorized actuator or fluidic lens of the optics 8 to change focal length, the optics 8 may have a manually adjustable focal length, e.g., a focus knob that is adjustable by the user's fingers. The user's indication of the target coming into focus may also be a manual input, e.g., as a button pressed by the user.

In another aspect of the method of FIG. 4, the processor 6 will process eye tracking data, produced by an eye tracking subsystem in the device 2, to determine whether the user's eye is looking at the target, and in response the processor will signal the alignment mechanism to perform the alignment process.

While certain aspects have been described and shown in the accompanying drawings, it is to be understood that such are merely illustrative of and not restrictive on the invention, and that the invention is not limited to the specific constructions and arrangements shown and described, since various other modifications may occur to those of ordinary skill in the art. For example, although the processor 6 may also be integrated within the device housing (along with the sensor 3, the display 7, the alignment mechanism 4 and the optics 8) in some instances part of the functionality or operations performed by the processor 6 can be performed by another processor that is in wired or wireless communication with the processor that is in the device housing. The other processor may be that of a laptop computer, a tablet computer, a smartphone, or even a website server. The description is thus to be regarded as illustrative instead of limiting.

What is claimed is:

1. A device for measuring properties of an eye, the device comprising:
a device housing;
a sensor in the device housing to measure a property of an eye of a user;
an electronically controlled alignment mechanism to which the sensor is coupled;
a display in the device housing that shows a target;
optics in the device housing and positioned in an optical path taken by the target, as shown by the display, from the display to the eye of the user, wherein the optics is configured to change accommodation by the eye, and has adjustable focal length that enables the user to see the target with changing acuity; and
a processor configured to signal the electronically controlled alignment mechanism to align the sensor to the eye of the user, in response to obtaining an indication that the eye of the user is focused on the target.

2. The device of claim 1 wherein the device housing is that of a handheld device that is to be held in a hand of the user while the user brings the device into proximity of the eye of the user, wherein the proximity is a distance, between the device housing and the eye, at which the sensor can measure pressure or take an image of the eye while the eye of the user can see the target being shown by the display.

3. The device of claim 1 wherein the target is a static image or an active image.

4. The device of claim 2 wherein the display is positioned no more than two hundred millimeters from the eye, when the device housing is in proximity of the eye.

5. The device of claim 4 wherein the optics comprises a convex solid lens that is moveable.

6. The device of claim 5 wherein the convex solid lens is motorized to be moveable as controlled electronically by the processor.

7. The device of claim 4 wherein the optics comprises a fluidic lens whose shape is controlled electronically by the processor.

8. The device of claim 4 wherein the processor is configured to obtain the indication that the eye of the user is focused on the target, by prompting the user to indicate when the user can see the target clearly, while the processor is signaling the optics to change the adjustable focal length.

9. The device of claim 8 further comprising a microphone in the device housing, wherein the processor processes an audio signal output by the microphone to detect audible input from the user as the indication that the user can see the target clearly.

10. The device of claim 1 wherein the processor is configured to obtain the indication that the eye of the user is focused on the target, by receiving manual or audible input from the user that the user can see the target clearly, wherein the optics can be manually adjusted by the user until the user can see the target clearly.

11. The device of claim 1 further comprising an eye tracking subsystem in the device housing that tracks the eye of the user, wherein the processor is configured to process eye tracking data, produced by the eye tracking subsystem, to determine whether the user is looking at the target, and in response signal the electronically controlled alignment mechanism.

12. A method for measuring properties of an eye using a device, the method comprising operations performed by a programmed processor as:
signaling a display in the device to show a target, wherein the device comprises optics positioned in an optical path taken by the target, as shown by the display, from the display to an eye of a user, the optics being configured to change accommodation by the eye and having adjustable focal length that enables the user to see the target with changing acuity;
obtaining an indication that the eye of the user is focused on the target, and in response signaling an alignment mechanism in the device to align a sensor in the device to the eye of the user; and
after signaling the alignment mechanism to align the sensor to the eye of the user, obtaining sensor data, produced by the sensor, which measures a property of the eye.

13. The method of claim 12 wherein obtaining the indication that the eye of the user is focused on the target comprises the programmed processor prompting the user to indicate when the user can see the target clearly, while the programmed processor is signaling the optics to change the adjustable focal length.

14. The method of claim 13 wherein obtaining indication that the eye of the user is focused on the target comprises the programmed processor processing an audio signal output by a microphone in the device, to detect audible input from the user as the indication that the user can see the target clearly.

15. The method of claim 12 wherein obtaining the indication that the eye of the user is focused on the target comprises the programmed processor receiving manual or audible input from the user that the user can see the target clearly, after the user has manually adjusted the optics until the user can see the target clearly.

16. The method of claim 12 further comprising the programmed processor processing eye tracking data produced by an eye tracking subsystem in the device that tracks the eye of the user, to determine whether the user is looking at the target, wherein signaling the alignment mechanism in the device to align the sensor is in response to having determined that the user is looking at the target.

17. The method of claim 12 wherein the target is a static image or an active image.

18. The method of claim 17 wherein the display is positioned no more than two hundred millimeters from the eye, when the device housing is in proximity of the eye.

19. A memory having stored therein instructions that configure a processor to perform a method comprising:
signaling a display in a device to show a target, wherein the device comprises optics positioned in an optical path taken by the target, as shown by the display, from the display to an eye of a user, the optics being configured to change accommodation by the eye and having adjustable focal length that enables the user to see the target with changing acuity;
obtaining an indication that the eye of the user is focused on the target, and in response signaling an alignment mechanism in the device to align a sensor in the device to the eye of the user; and
after signaling the alignment mechanism to align the sensor to the eye of the user, obtaining sensor data, produced by the sensor, which measures a property of the eye.

20. The memory of claim 19 having stored therein further instructions that configure the processor to process eye tracking data produced by an eye tracking subsystem in the device that tracks the eye of the user, to determine whether the user is looking at the target, wherein the processor signaling the alignment mechanism in the device to align the sensor is in response to the processor having determined that the user is looking at the target.
